(19) United States
(12) Patent Application Publication
(10) Pub. No.: US 2022/0062174 A1
(43) Pub. Date: Mar. 3, 2022

(54) Therapeutic Medical Device and Methods of Use

(71) Applicant: KeriCure Inc., Wesley Chapel, FL (US)

(72) Inventor: Kerriann Robyn GREENHALGH, Tampa, FL (US)

(21) Appl. No.: 17/412,231

(22) Filed: Aug. 25, 2021

Related U.S. Application Data
(60) Provisional application No. 63/069,856, filed on Aug. 25, 2020.

(51) Int. Cl. A61K 9/107; A61K 33/38; A61K 45/06; A61K 47/32

(52) U.S. Cl. CPC A61K 9/1075; A61K 33/38; B82Y 5/00; A61K 47/32; A61K 45/06

(57) ABSTRACT

A micro-emulsion compound, especially suited for topical applications, comprising a nanopolymer, an anesthetic agent such as lidocaine, and particles of biologically active silver, silver oxide complex, or another biologically active metal.

### RELATED APPLICATIONS

This application claims priority from U.S. Provisional Application No. 63/069,856, filed Aug. 25, 2020, for “Therapeutic Medical Device and Methods of Use” by Kerriann Robyn Greenhalgh, which is incorporated by reference herein in its entirety. In addition, all documents and references cited herein and in the above referenced applications, are hereby incorporated herein by reference.

### FIELD OF THE DISCLOSURE

The present invention is directed to polyacrylate compounds, and more particularly to polyacrylate nanopolymer compounds containing nano-sized silver particles in combination with a therapeutic drug in a hydrogel-like system for application to biological tissues.

### BACKGROUND OF THE INVENTION

Many elemental metals have been used throughout history to treat infections and disease. Many cultures have relied on elemental copper to reduce inflammation, zinc has long been used as an antiviral and topically in wounds to help promote healing, and lithium is widely accepted as a prophylaxis to treat manic-depressive disorder. Silver has been widely recognized in the medical community as a safe and effective alternative to traditional antibiotics and antifungal drugs for topical treatment of infections.

Silver containing hydrogels, ointments and dressings have made a significant impact on the wound care market over the past two decades and are widely accepted among physicians as providing many positive benefits for the patient. However, there are many drawbacks to these products.

The majority of the topical silver hydrogels and ointments require direct contact with the patient for the material to be applied. This is detrimental for two reasons. First, the direct contact between the medical professional and the patient's wound creates the risk for nosocomial infection. Second, the physical spreading of the material over the wound can be extremely painful for the patient.

In addition, many of the products are not clear but white in color, which limits the visibility to the wound bed and hinders the medical staff from accurately judging the progression of wound healing. Therefore, the product must be removed so the medical staff can evaluate the wound's progress, which is also often a painful and detrimental procedure.

Silver loaded bandages and dressings also require direct contact with the wound for application and require frequent dressing changes, which not only can be painful for the patient, but can also cause damage to freshly forming epithelial layers, ultimately setting back the healing process with each dressing change.

Further, despite the beneficial effects of various biologically active metals for wound care or other medical treatments, they do not have an immediate effect on a patient's pain, either from the initial wound or trauma or pain resulting from treatment such as dressing changes.

What is needed is an antimicrobial/antiviral product containing silver and/or another biologically active metal together with an anesthetic agent that overcomes the disadvantages of the prior art.

### SUMMARY OF THE INVENTION

Embodiments of the present invention provide a micro-emulsion compound, especially suited for topical applications, comprising a nanopolymer, an anesthetic agent such as lidocaine, and particles of biologically active silver, silver oxide complex, or another biologically active metal.

In one aspect of the invention, the micro-emulsion compound is a liquid that forms a solid layer when applied to a surface such as biological tissue. Embodiments of the present invention additionally provide an anesthetic agent, especially suited for topical applications, comprising drug agents such as lidocaine, benzocaine, borneol, bupivacaine, and/or their derivatives, wherein the drug is dispersed in a solution, suspension, emulsion, or micro-emulsion containing nanopolymer particles and nanosilver particles.

In one aspect of the invention, a micro-emulsion compound comprises a nanopolymer; an anesthetic agent; and particles of biologically active silver; wherein the micro-emulsion compound is a liquid that forms a solid layer when applied to a surface.

In another aspect of the invention, a compound for topical treatment of wounds or biological tissues comprises a polyacrylate polymer; an anesthetic agent; and particles of a biologically active metal; wherein the biologically active metal particles are surrounded by an outer layer of a polyacrylate polymer and dispersed in a solution, suspension, emulsion, or micro-emulsion.

In another aspect of the invention, a method of preparing a biologically active micro-emulsion for topical application to biological tissues is provided, the method comprising combining one or more acrylate monomers in purified water, a surfactant, and a water-soluble radical initiator to form a solution, suspension, emulsion, or micro-emulsion; and then adding a silver nanoparticle solution and a solution containing an anesthetic agent to the solution, suspension, emulsion, or micro-emulsion.

In another aspect of the invention, a method of preparing a microemulsion comprising biologically active silver and an anesthetic agent is provided, the method comprising forming a homogeneous solution of one or more acrylate monomers, introducing a silver and anesthetic constituent to the solution while in a hydrophilic solvent, said silver constituent comprising biologically active silver, and said anesthetic agent comprising lidocaine, adding a surfactant to the suspension to form micelles of acrylate monomer surrounding particles of the silver constituent and entrapping the lidocaine, and polymerizing the polyacrylate monomers to form a suspension of silver particles and lidocaine encased in a polyacrylate nanoparticle through a radical initiator.

In another aspect of the invention, a method of preparing an antimicrobial and anesthetic compound for topical application to biological tissues is provided, the method comprising: combining an anesthetic agent with one or more acrylate monomers in purified water, a surfactant, and a water-soluble radical initiator to form a solution or micro-emulsion; and then combining a biologically active silver constituent in a hydrophilic solvent.

In another aspect of the invention, a method of preparing an antimicrobial and anesthetic compound for topical application to biological tissues is provided, the method comprising: combining a biologically active silver constituent in a hydrophilic solvent with one or more acrylate monomers, a surfactant, and a water-soluble radical initiator to form a biologically active solution or emulsion, then combining with an anesthetic agent dissolved in a solvent.

In another aspect of the invention, a method of preparing an antimicrobial and anesthetic compound for topical application to biological tissues is provided, the method comprising: combining one or more acrylate monomers in purified water, a surfactant, and a water soluble radical initiator to form a solution or micro-emulsion; and then combining a biologically active silver constituent in a hydrophilic solvent and a solution containing an anesthetic agent dissolved in a hydrophilic solvent. A hydrophilic solvent can comprise, for example, a solution including about 1-30% ethanol, specifically about 12-18% for the non-freezing capabilities.

In some embodiments, a gelling agent is combined with a micro-emulsion containing nanopolymer, nanosilver particles, and lidocaine. The gelling agent can comprise a hydrophilic solution containing a cellulose composition in a gel state, containing about 0.1% to about 5% cellulose.

In another aspect of the invention, a hydrophilic solution containing 1 to 100 ppm of nanosilver or silver tetraoxide is combined with a micro-emulsion containing nanopolymer particles and lidocaine.

In another aspect of the invention, a hydrophilic solution containing 0.1 to 5% of an anesthetic agent, such as lidocaine, benzocaine, borneol, bupivacaine, and/or their derivatives, is combined with a micro-emulsion containing nanosilver and nanopolymer particles.

In another aspect of the invention, a method of treating a wound using any of the compositions described herein is provided.

The foregoing has outlined rather broadly the features and technical advantages of the present invention in order that the detailed description of the invention that follows may be better understood. Additional features and advantages of the invention will be described hereinafter. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION OF THE INVENTION

Hydrogel wound dressings are an established category of advanced wound care products that have very specific applications in wound care. The benefits of hydrogels are predominantly seen in chronic wound care and burn wound care, where maintaining the proper levels of moisture within the wound is a critical aspect for wound healing. These dressings also frequently have agents incorporated that aid in autolytic debridement, which provides an enzymatic or chemical method of disrupting necrotic tissue within a wound and breaking down the tissue for removal without the use of mechanical disruption to the wound bed. Some hydrogel products also contain ionic solutions of silver salts as a preservative to kill microbes within the dressing.

Silver containing wound dressings, hydrogels, ointments, and dressings have made a significant impact on the wound care market over the past two decades and are widely accepted among physicians as providing many positive benefits for the patient. These wound dressings often employ silver salt compounds, such as silver nitrate, silver arsphenamine or silver chloride salts, which have a long history of negative side effects, including allergic reactions, toxicity, and a phenomenon known as argyria or blue skin effect. Elemental silver particles or silver complex particles have not been used in prior art wound dressings due in part because such particles are considerably more expensive than silver salt compounds and have limited commercial availability from major chemical suppliers.

However, Applicant has discovered that the benefits of elemental or complex silver nanoparticles are significantly superior to these silver salt chemicals, with little to no reported side effects from ingestion or topical use. Elemental silver in nanoparticle form has demonstrated anti-inflammatory benefits as well as significant antimicrobial activity, especially against drug resistant strains of bacteria including methicillin resistant S. aureus (MRSA) and vancomycin resistant enterococcus (VRE) microbes. Further, there has been no reported development of resistance development to the nanoparticle silver to date. Therefore, the use of nanoparticle silver, according to embodiments of the invention, is a highly effective antimicrobial agent that can provide superior activity in a topical product, including a wound dressing or a hydrogel, over currently marketed products containing ionic silver or silver salt chemicals, especially when combined with an anesthetic drug as described below.

Lidocaine is one example of an anesthetic agent that has been available as an OTC drug or prescription strength in a number of commercial products for itching, numbing, and pain relief. Lidocaine works as a localized nerve blocking agent when applied topically and is commonly applied as a gel to the skin surface to numb the area prior to a procedure. However, adoption of the anesthetic in advanced wound care has not been seen to date. While commonly used by physicians prior to dressing changes and wound debridement to numb the sensitive tissue and reduce the pain associated with these procedures, the drug has not been incorporated in an advanced wound care product to date.

According to embodiments of the present invention, compositions can include a nanopolymer emulsion containing biologically active metallic nanoparticles and an anesthetic agent that provides unique attributes which enhance and complement one another as a medical device and provide additional benefits outside of those typically associated with medical products containing silver or anesthetic agent alone. In some embodiments, such compositions can comprise a homogeneous suspension of nanoparticles, wherein said nanoparticles contain an outer layer of a non-degradable polymer coating and an inner core of elemental silver or silver tetraoxide and an anesthetic drug such as lidocaine, benzocaine, borneol, or bupivacaine. In some embodiments, the nanoparticle silver element is incorporated within the nanopolymer shell, and the anesthetic is in free form in the suspension. In some embodiments, the anesthetic is entrapped within the nanopolymer, which is suspended in a micro-emulsion containing silver nanoparticles. In other embodiments, a micro-emulsion of nanopolymer is combined with solutions of silver nanoparticles and an anesthetic agent in a homogeneous mixture. In addition, compositions can have additional metal-containing components added to the suspension that provide additional and/or uniquely different biological activity than the core metal within the nanoparticles. Additional additives and metal particles can be added to the composition at various stages of the microemulsion process for additional biological benefits.

A micro-emulsion compound as described herein can be a liquid that forms a solid or semi-solid sheet or film that conforms to a surface on which it is applied. In some embodiments, the applied liquid can form such a solid or semi-solid sheet or film in less than about 30 minutes, such as less than about 10 minutes, such as less than 5 minutes, less than 3 minutes, or less than one minute.

Upon application to a biological tissue or medical device, some embodiments of the invention donate moisture to the biological tissue that permits an optimal level of moisture within the tissue. In some embodiments, moisture levels are further controlled by the formation of a solid or semi-solid sheet or film that conforms to the surface on which it is applied.

The solid material formation is the result of the solvent portion of the suspension being removed either through evaporation or absorption into a tissue or other absorbent material, such as foam, gauze, or other porous material. In this solid form, the embedded silver nanoparticles and anesthetic agent are capable of eliciting similar biological behaviors as when applied individually to the biological tissue in a solvent or gel system. Additionally, the effects of the solvent system may provide longer lasting effects and deeper penetration of these biological agents into the biological tissue that are not established in the individual systems without the incorporation of the micro-emulsion system.

In addition, in some embodiments, the solid form of the composition has the ability to limit contamination of the underlying biological tissue through the physical barrier nature of the solid film and the composition's ability to entrap a portion of the silver nanoparticles within the solid film to provide antimicrobial activity at the surface of the treated tissue.

In addition, compositions according to embodiments of the invention, when in either morphology (liquid or solid polymer film) have unique properties that make them desirable for the treatment of bacterial, fungal, and viral infections. The compositions also have benefits that include but are not limited to the management of acute pain, inflammation, and irritations.

The unique nanoparticle formulations described herein provide a number of advantages over the various wound dressings and hydrogel products available. First is the ability to provide a highly stable combination of silver nanoparticles and an anesthetic in a water-based system. Second is the ability to incorporate highly water insoluble anesthetic agents, including lidocaine, in a water-based system. While lidocaine hydrochloride is the commonly used salt form of lidocaine that permits solubilization in a hydrophilic system, the composition described here embodies lidocaine in the non-salt, USP grade form, which is more stable, has a higher activity profile, and has a better safety profile than the hydrochloride form. This form of lidocaine is not used in the prior art due to the insolubility in hydrophilic systems that are required for use in medical devices. The invention described herein has overcome this obstacle with the micro-emulsion system and combination of hydrophilic solvents—such as an alcohol and water combination as described below—that permits homogeneous suspension in the solution.

Colloidal and nanoparticle silver formulas exhibit difficulties remaining suspended in solution. Silver colloids especially have a tendency to aggregate when suspended and settle, causing early precipitation of the silver from the suspension. Embodiments of the present invention provide a means of suspending the silver particles within a stable nanopolymer system, donating its stability benefits to the suspended nanoparticle silver.

Additionally, nanoparticle or colloidal silver solutions in the prior art are unable to undergo freezing and thawing processes. If frozen, these suspensions are broken, and the silver is no longer soluble within the water-based system. In the present composition, the use of a hydrophilic solvent such as a mixture of alcohol and water results in a micro-emulsion capable of multiple freeze-thaw cycles without detriment to the silver nanoparticles or the nanopolymer suspension. This is a new and unique feature of the hydrogel composition according to some embodiments of the invention that is credited to the method by which the anesthetic is incorporated.

The compositions described herein have applications throughout wound care as an advanced wound dressing and/or hydrogel style product. The duality of the composition as a water-based liquid that transforms into a solid dressing upon application provides usage in many areas of wound care. Additionally, the combination of antimicrobial and anesthetic agents embedded within the hydrogel-style wound dressing afford benefits to the wound that permit rapid healing with lower strain on the individual. Wounds that can be treated with the composition include poly-wounds, open fracture wounds, all forms of burn wounds, chemical agent related wounds, including nerve agent, chemical agent, and chemical-induced wounds, chronic wounds including pressure injuries, diabetic ulcers, non-healing wounds, lacerations, abrasions, surgical wounds, excisions, minor wounds including cuts and scrapes, infected wounds, and non-infected wounds. The composition can also be combined with other advanced wound care materials including skin grafts of human, animal or synthetic nature, secondary dressings, disinfecting agents for microbial or chemical disinfecting, tourniquet, and other blood loss control compositions.

Embodiments of the present invention provide numerous benefits over the prior art as the combinational aspects of the invention overcome many shortcomings of the prior art. First, as a wound dressing, the ability to utilize a dispenser mechanism, such as a spray, dropper, or ampule applicator, to administer the wound dressing to any size or shape wound, anywhere on the body, provides a number of benefits over wound dressings that are comprised of a sheet material and therefore are limited in their scope of application. Further, these dressings are often non-transparent, or become non-transparent quickly once applied to the wound bed as interactions with wound exudates cloud the dressings. The composition as described in the invention is a milky white color when in its fluid state, but turns completely clear when dried, which allows full visibility of the wound throughout the treatment process. Additionally, the solid film that forms is a thin, highly elastic film that can stretch and move with the body, allowing a full range of motion for the patient while maintaining its structural integrity and remains bound to the exposed tissue in extreme environments. Another advantage of the composition presented is the ability to layer the composition through multiple applications to the surface of the wound bed. Prior art wound dressings require frequent to daily removal and reapplication for proper healing and to monitor healing progress. Even prior art that are in a cream or ointment form require daily removal and reapplication as the materials can cake up or become unmanageable when left on a wound for longer than 1 to 2 days. Embodiments of the present invention significantly reduce the need for dressing changes, with re-application of the invention required every three days or greater, without the need to remove the invention during the dressing change. This is a substantial benefit to medical staff charged with managing wound treatment, especially for wounds where dressing changes are problematic, such as chronic wounds and burn wounds, where direct contact with the wounds can be painful and increases the exposure to pathogens that can retard healing.

In the instances where a deeply penetrating infection is recognized within a tissue, the liquid delivery method of the wound dressing affords antimicrobial activity in deep pockets of wounds where infections can establish and advance to biofilms if not properly treated. The unique combination of nanoparticle silver delivery with simultaneous barrier formation over the surface of the wound, even in the deep pockets, provides superior antibacterial protection not afforded by the prior art. Equally, the delivery of anesthetics such as lidocaine to the wound provides fast absorption and localized pain management that is not available in the prior art.

According to some embodiments, compositions are provided comprising incorporation of a specific ratio of solvents—a mixture of about 5% to about 50% alcohol (IPA, ethanol, tertbutyl alcohol) in water, more specifically about 10% to about 20% ethanol in water—provide attributes not established in the prior art. These attributes include the ability of the invention to be stable in extreme environments, such as freezing temperatures, without degradation or destruction to the micro-emulsion. It also affords the invention to have a faster setting time than the prior art, wherein after application to a wound bed, the invention transforms from the liquid form to the solid dressing in less time than the prior art. This advancement in the current invention provides a superior application method over the prior art, where reduced time for application is a critical parameter for treatment of wounds, especially in critical care, emergency, and field care uses of the invention.

Some embodiments of the invention also exhibit attributes of the solid form that vary from the prior art, including a thinner solid film that forms which is permits greater permeation of gasses such as oxygen, or in layman's terms, the wound barrier is more breathable over the wound.

Some embodiments of the invention also include compositions where a gelling agent is formulated within the invention. The gelling agent is specific to the invention as the micro-emulsion system is not compatible with most chemical compositions that create a gel or hydrogel system. The gelling agent in the present invention provides additional moisture donation to the wound bed that far exceeds that of the prior art and compositions that do not include the gelling agent.

Some embodiments of the invention provide compositions that permit absorption of fluids, including exudates from wound beds. While the ability of a wound dressing to absorb exudates is not novel, one that contains embedded nanoparticles silver and lidocaine, in a spray on dressing that is also moisture-donating, not just moisture absorptive, significantly differs from the prior art.

Some embodiments provide compositions that can coat other wound healing mechanisms, including skin grafts, absorptive foam dressings, and can act as a bridge between peripheral skin and a dressing or device, such as an ostomy bag. negative pressure wound dressing or fill the gap between a protruding medical device such as a bone pin or screw and the wound bed. damaged tissue and in between intact tissue and foreign objects such as an ostomy bag or a protruding medical pin or screw. Application between the skin and foreign materials can aide in preventing the deterioration of the skin in that area and reduce the risk of infection. Wound management, skin care, infection control and prevention, among other things, can be significantly simplified with use of embodiments of the present invention.

In this application, the term “compositions” will generally refer to any solution, suspension, emulsion, micro-emulsion, or other mixture according to any of the embodiments described herein. Further, as used herein, the term “applying”, in the context of compositions according to embodiments of the present invention, means contacting the composition on, in, and/or around a desired anatomical site, such as a wound or an unwounded site on or in the body. Such compositions can be applied to any intact or wounded, hard or soft tissue of the body (e.g., connective, muscle, nervous epithelial, or combination of two or more types). The composition is kept in contact with the anatomical site to achieve a desired result, such as protection of the site, promoting wound healing, and delivering the biologically active silver agent.

The following are examples that illustrate procedures for practicing embodiments of the invention. These examples should not be construed as limiting. All percentages are by weight and all solvent mixture proportions are by volume unless otherwise noted.

A biologically active silver-based nanoparticle suspension according to embodiments of the invention can utilize three unique constituents in the formulation: a) a silver component, composed of neutral or positively charged silver particles, b) one or more acrylate monomers, such as butyl acrylate, methyl acrylate, ethyl acrylate, methyl methacrylate, methacrylate, styrene, phenyl acrylate, or methacrylamide, and c) a natural or biologically enhancing additive.

In a specific embodiment, a polyacrylate coating is formed around silver particles in a single step during a microemulsion polymerization process, wherein the polyacrylate content can be approximately 0.01% to 30% (w/w) of the total formulation, such as approximately 5% to 25%, approximately 25%, approximately 20%. For some uses, it is desirable to reduce the solid content to less than 0.1%. Additionally, when two acrylate monomers are employed, these monomers can be incorporated in a ratio of about 9:1, 8:2, 7:3, 6:4 or 5:5 of a base acrylate to supporting acrylate monomer. Suitable acrylate monomers are described in U.S. Pat. App. No. 2014/0004204 by Greenhalgh et al. for “Biocompatible polyacrylate compositions and methods of use” (filed Jun. 18, 2013), which is hereby incorporated by reference. Concentrations described herein, unless otherwise stated, will be based upon the weight of a component compared to the weight of the total solution.

Silver content can be incorporated into the microemulsion process in the form of solubilized silver in purified water, wherein the silver containing water can be approximately 50% to approximately 99.99% (w/w), such as 70% of the total solution, and can contain additional salts and/or buffers. The silver content can comprise biologically active silver, such as for example colloidal, nanocrystalline, ionically charged silver particles, or nanoparticle silver.

To create the micelle precursors in the solution, 0.1-5% of an anionic surfactant can be used. As would be recognized by persons of skill in the art, an anionic surfactant is a macromolecule, usually in the sulfonate or sulfate group of chemicals such as sodium lauryl sulfate, which acts as an active surface agent to lower the surface tension of liquids and permit stable interactions between hydrophobic and hydrophilic elements. Suitable surfactants include, for example, lauryl alcohol, sodium dodecyl sulfate, lechitin, lauryl sulfate sodium salt, sodium dodecylbenzene sulphonate, sodium dioctyl sulphosuccinate, unsaturated sodium or potassium salts of fatty acids, and/or saturated sodium or potassium salts of fatty acids.

Additionally, a radical initiator of about 0.1% to about 2% can be used to initiate the polymerization. Typical examples of radical initiators include halogen molecules, azo compounds, peroxides, alkyl hydroperoxides, sodium salt of persulphate, ammonium salt of persulphate, potassium salt of persulphate, thiosulphates, metabisulphites, and hydrosulphides.

The following steps can be used to make a microemulsion suspension according to a specific embodiment. First, the acrylate(s) are measured according to the total weight of the formulation, then mixed until homogeneous at about 50° C. to about 95° C. Thereafter, surfactant and the hydrophilic solvent, consisting of water or a combination of water and nanoparticle silver suspended in water, are added to the mixture, and vigorously stirred in order to create micelles. Upon successful micelle formation, the radical initiator is added to the system and an oxygen free environment is created to allow a radical polymerization process to occur. The solution is mixed for 5-10 hours to completion.

In some embodiments, additional silver functionality can be added post polymerization by direct addition of the desired silver solution to the micro-emulsion by agitation, sonication, or other methods to permit interaction between the silver particles and the nanopolymer surface.

Additives can also be incorporated into the hydrophobic inner core of the nanopolymer particles by dissolving the additive within the acrylate monomer(s) during the initial micelle phase for solubilization. In this embodiment, the additives are trapped within some of the nanopolymer particles. Whereas additives can also be added prior to the microemulsion process or post polymerization, either directly or when dissolved in a hydrophilic solvent, with gentle mixing until homogeneous. In some embodiments, up to approximately 50% of the solid content of the micro-emulsion can consist of additives including the silver nanoparticles and anesthetic agents.

In other embodiments, the additives are incorporated using a mixture of an alcohol and water system, at a percentage of about 1% to about 30% alcohol in water, such as 10% to 20% alcohol in water, to dissolve the additive prior to addition to the micro-emulsion system, either during the micelle formation stage, or post polymerization.

The invention described herein has broad applicability and can provide many benefits as described and shown in the examples above. The embodiments will vary greatly depending upon the specific application, and not every embodiment will provide all of the benefits and meet all of the objectives that are achievable by the invention. Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention. After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

All patents, patent applications, provisional applications, and publications referred to or cited herein are incorporated by reference in their entirety, including all figures and tables, to the extent they are not inconsistent with the explicit teachings of this specification.

In the discussion and in the claims, the terms “including” and “comprising” are used in an open-ended fashion, and thus should be interpreted to mean “including, but not limited to . . . .” Further, unless expressly stated to the contrary, “or” refers to an inclusive-or and not to an exclusive-or. Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise. To the extent that any term is not specially defined in this specification, the intent is that the term is to be given its plain and ordinary meaning. The accompanying drawings are intended to aid in understanding the present invention and, unless otherwise indicated, are not drawn to scale.

Although the present invention and its advantages have been described in detail, it should be understood that various changes, substitutions, and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps. The figures described herein are generally schematic and do not necessarily portray the embodiments of the invention in proper proportion or scale.

What is claimed is:

1. A micro-emulsion compound comprising:
a nanopolymer;
an anesthetic agent; and
particles of biologically active silver;
wherein the micro-emulsion compound is a liquid that forms a solid layer when applied to a surface.

2. The micro-emulsion compound of claim 1 in which the micro-emulsion is formed through a micro-emulsion polymerization process.

3. The micro-emulsion compound of claim 1 in which the applied liquid micro-emulsion compound forms a solid layer on the surface in less than 5 minutes.

4. The micro-emulsion compound of claim 1 in which the nanopolymer is a polyacrylate polymer.

5. The micro-emulsion compound of claim 4, wherein said polyacrylate polymer is a combination of acrylate monomers comprising butyl acrylate, ethyl acrylate, methyl acrylate, methyl methacrylate, methacrylamide, phenyl acrylate, and/or styrene.

6. The micro-emulsion compound of claim 1 in which the particles of biologically active silver comprise nanoparticles of silver.

7. The micro-emulsion compound of claim 6 in which the nanoparticles of silver are comprised of elemental silver, silver particles, and/or silver tetraoxide.

8. The micro-emulsion compound of claim 6 in which the micro-emulsion is formed through a micro-emulsion polymerization process and the nanoparticles of silver are encapsulated by the nanopolymer during the polymerization process.

9. The micro-emulsion compound of claim 6 in which the micro-emulsion is formed through a micro-emulsion polymerization process and the nanoparticles of silver are incorporated after the polymerization process of the micro-emulsion has completed.

10. The micro-emulsion compound of claim 1 in which the anesthetic agent comprises lidocaine, benzocaine, borneol, bupivacaine, and/or their derivatives.

11. The micro-emulsion compound of claim 2 in which the anesthetic agent is incorporated during the polymerization process of the micro-emulsion.

12. The micro-emulsion compound of claim 2 in which the anesthetic agent is dissolved in the micro-emulsion prior to the polymerization process.

13. The micro-emulsion compound of claim 11 in which the anesthetic agent is encapsulated within the nanopolymer particles in the micro-emulsion.

14. The micro-emulsion compound claim 2 in which the anesthetic agent is incorporated after the polymerization process of the micro-emulsion is completed.

15. The micro-emulsion compound of claim 1 wherein the anesthetic agent is lidocaine and is dissolved in a solvent prior to addition to the micro-emulsion.

16. The micro-emulsion compound of claim 1 in which the nanopolymer, anesthetic agent, and particles of biologically active silver are suspended in a hydrophilic solvent.

17. The micro-emulsion compound of claim 16 in which the hydrophilic solvent comprises a mixture of water and ethyl alcohol, isopropyl alcohol, and/or tertbutyl alcohol.

18. The micro-emulsion compound of claim 1 in which the micro-emulsion compound is capable of reconstituting after freezing or boiling.

19. The micro-emulsion compound of claim 1 further comprising a plant or animal based biological constituent including collagen, gelatin, proteins, hormones, and/or cellulose.

20. The micro-emulsion compound of claim 1 further comprising an additive that provides beneficial attributes that enhance wound healing.

21. A method of preparing a biologically active micro-emulsion for topical application to biological tissues, the method comprising:
combining one or more acrylate monomers in purified water, a surfactant, and a water-soluble radical initiator to form a solution, suspension, emulsion, or micro-emulsion; and
then adding a silver nanoparticle solution and a solution containing an anesthetic agent to the solution, suspension, emulsion, or micro-emulsion.

22. The method of claim 21 in which the silver nanoparticle solution comprises silver oxide, metallic silver, nanocrystalline silver, silver hydrosol, and/or colloidal silver.

23. A compound for topical treatment of wounds or biological tissues comprising:
a polyacrylate polymer;
an anesthetic agent; and
particles of a biologically active metal;
wherein the biologically active metal particles are surrounded by an outer layer of a polyacrylate polymer and dispersed in a solution, suspension, emulsion, or micro-emulsion.
